(19) United States
(12) Patent Application Publication
(10) Pub. No.: US 2025/0235624 A1
(43) Pub. Date: Jul. 24, 2025

(54) Injection Device and Method of Unlocking Operation of the Injection Device

(71) Applicant: Sanofi, Paris (FR)

(72) Inventors: Stefan Alt, Frankfurt am Main (DE); Tim Glässer, Rüsselsheim (DE); Michael Helmer, Frankfurt am Main (DE); Stephan Mücke, Rüsselsheim (DE); Peter Nober, Rüsselsheim (DE); Michael Schabbach, Frankfurt am Main (DE); Martin Vitt, Frankfurt am Main (DE)

(21) Appl. No.: 18/700,964

(22) PCT Filed: Nov. 1, 2022
(86) PCT No.: PCT/EP2022/080409

§ 371 Date: Apr. 12, 2024

(30) Foreign Application Priority Data
Nov. 3, 2021 (EP) 21315210.1

(51) Int. Cl. A61M 5/315; A61M 5/50

(52) U.S. Cl. CPC A61M 5/31546; A61M 5/31571; A61M 5/31585; A61M 5/5086; A61M 2205/3317; A61M 2205/3569; A61M 2205/52; A61M 2205/6009

(57) ABSTRACT

An injection device comprises a housing of elongated shape and extending along a longitudinal direction, wherein the housing comprises a distal housing component and a proximal housing component, wherein the distal housing component is configured to accommodate a medicament container and wherein the proximal housing component is configured to accommodate or to support a drive mechanism to operably engage with the medicament container for expelling or withdrawing of the dose of the medicament from the medicament container, the drive mechanism to operably engage with the medicament container, an electronic unit coupled to the drive mechanism, an electric sensor provided at or near a distal end of the proximal housing component, electrically connected to the electronic unit and configured to contactlessly detect an electric identifier provided on or in one of the medicament container and the distal housing component.

### CROSS-REFERENCE TO RELATED APPLICATIONS

The present application is the national stage entry of International Patent Application No. PCT/EP2022/080409, filed on Nov. 1, 2022, and claims priority to Application No. EP 21315210.1, filed on Nov. 3, 2021, the disclosures of which are incorporated herein by reference.

### TECHNICAL FIELD

The present disclosure relates to an injection device, such as a pen-type injector and to a method of unlocking operation of the injection device, e.g. by reading or capturing of an electric or electronic identifier provided on or associated with a medicament container. In further aspects the disclosure relates to a method of unlocking operation of the injection device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

With reusable injection devices a medicament container, i.e. a cartridge has to be replaced when the medicament has been depleted. Here, the medicament container, e.g. a vitreous cartridge filled with the medicament has to be replaced. Some primary medicament containers, e.g. in form of a cartridge filled with the medicament are preassembled in a housing component of the injection device, such as a cartridge holder.

In either way and with reusable devices it should be ensured, that only a dedicated medicament container or dedicated housing component preassembled with such a medicament container can be used with a dedicated drive mechanism of an injection device.

Generally, there may be provided a large variety of primary medicament containers that differ with regards to their filling volume or with regard to the pharmaceutical substance or concentration of the medicament. For replacement of a medicament container it must be ensured, that an injection device or drive mechanism can only be used in conjunction with a dedicated medicament container intended for use with this particular type of injection device or drive mechanism. Unintended cross use of non-matching pairs of medicament containers and drive mechanisms or injection devices has to be avoided.

Some approaches suggest to mechanically encode the primary container, e.g. the medicament container and/or the cartridge holder equipped with such a medicament container to prevent unintended mechanical connection to a non-matching drive mechanism or non-matching injection device. Such solutions require a redesign of respective mechanical components of the injection device. Providing a large variety of differently mechanically encoded housing components is rather elaborate in terms of manufacturing and manufacturing logistics of the injection device.

It is therefore intended to provide an improved injection device, and a method of operating an injection device aiming to enhance patient safety and to prevent unintended cross use of replaceable medicament containers with an injection device. The present solution aims to avoid a mechanical redesign of housing components of existing injection devices. It is desirable to provide a solution that is easily integrable into existing injection devices or injection systems. It should be easily adaptable to varying demands or varying scenarios of use of the injection device.

### SUMMARY

In one aspect there is provided an injection device for injecting of a dose of a medicament. The injection device comprises a housing of elongated shape and extending along a longitudinal direction (z). The housing comprises a distal housing component, e.g. denoted as a cartridge holder, and a proximal housing component, also denoted as a body. The distal housing component is configured to accommodate a medicament container. The proximal housing component is configured to accommodate or to support a drive mechanism to operably engage with the medicament container for expelling or withdrawing of the dose of the medicament from the medicament container. The distal housing component comprises a distal end facing away from the proximal housing component. The distal end of the distal housing component is typically provided or connected with an injection needle so as to pierce or to puncture biological tissue for injecting the medicament.

The injection device further comprises a drive mechanism arranged or assembled in the proximal housing component and configured to operably engage with the medicament container. Typically, the drive mechanism comprises a piston rod configured to apply a distally directed thrust onto a stopper or bung of the medicament container to expel a liquid medicament from the medicament container.

The injection device further comprises an electronic unit coupled to the drive mechanism. The injection device also comprises an electric sensor provided at or near a distal end of the proximal housing component. The electric sensor is electrically connected to the electronic unit and is configured to detect an electric identifier provided on or in one of the medicament container and the distal housing component. The electronic unit can be embedded in or on the injection device. It may be arranged inside the housing of the injection device and may be permanently connected to or assembled inside the injection device. With some examples the electronic unit is an embedded electronic unit being integrated into the injection device or its drive mechanism. The electrical connection between the electric sensor and the electronic unit as well as any other electrical connection mentioned herein may be implemented as a wired connection.

Typically and by way of an electric or electronic interaction between the electric sensor and the electric identifier information being indicative of the identifier and/or identifier information being indicative of the medicament container can be captured, read and/or processed by the electronic unit of the injection device.

The electric sensor is particularly configured to contactlessly detect the presence and/or the type of the electric identifier, which may be provided on or in one of the medicament container and the distal housing component. Such a contactless electric detection may include an inductive coupling between the electric sensor and the electric identifier. The contactless detection of the electric identifier by the electric sensor is beneficial in that the proximal housing component and the distal housing component may be void of electric contact pins or electric contact pads to provide an electrical connection between the distal and proximal housing component.

Typically, the electric identifier can be implemented as an electronic identifier, as an electronic passive identifier and/or as an inductive identifier.

Moreover, a contactless, e.g. inductive coupling between the electric identifier and the electric sensor provides and enables a rather universal design of the proximal housing component and the distal housing component. The electric sensor and the electric identifier may be invisible from outside the housing. They may be located on an inside of the housing, e.g. on an inside of a sidewall of the housing or they may be embedded in a sidewall of the respective housing component. This way, a rather long-lasting, robust and reliable implementation of an electric sensor and an electric identifier can be provided.

The electric identifier provides an electric encoding of at least one of the distal housing component and the medicament container. Distal housing components and/or medicament containers of different type may distinguish by electrically or electronically measurable parameters of the respective electric identifier. By way of implementing an electrically encoded medicament container or distal housing component the mechanical connection interface between the distal housing component and the proximal housing component can remain unaltered for all available types of different housing components and/or medicament containers.

According to a further example the drive mechanism comprises at least one interlock configured to disable at least one of setting of the dose and dispensing of the dose. Here, and based on signals obtained from the electric sensor the at least one interlock can be at least temporarily deactivated. When the information or data which is indicative of the identifier matches with predefined medicament container related data, e.g. stored in the electronic unit, the drive mechanism will be unlocked and the respective medicament container can be used with the drive mechanism of the injection device.

With some examples, wherein the electric identifier as provided on or in one of the distal housing component and the medicament container does not match with the drive mechanism and wherein the information obtained from the electric identifier by the electric sensor does not match with predefined medicament container related data the interlock remains activated and use of the injection device, e.g. for the purpose of setting and/or dispensing of the dose is impeded or blocked.

Signal processing may be conducted by the electronic unit of the injection device in order to keep the interlock activated, e.g. when the electric identifier does not match with a predefined requirements, demands or scenarios of use. The electronic unit may keep the at least one interlock activated so that at least one of setting and dispensing of the dose is and remains disabled. When the data obtained from the electric identifier matches predefined requirements, demands or scenarios of use the electronic unit may at least temporarily deactivate the at least one interlock and the drive mechanism becomes unlocked for providing at least one of setting and injecting of the dose of the medicament.

According to a further example the electronic unit of the injection device is operable to control the at least one interlock on the basis of signals received from the electric sensor. If signals received from the electric sensor indicate the presence of a medicament container of a first type while the electronic unit is embedded in a drive mechanism of a second type, which is generally incompatible with the medicament container of the first type, the electronic unit is operable to activate the interlock so as to impede at least one of setting and injecting of a dose of the medicament.

With a further example, the proximal end of the distal housing component is detachably connectable to a distal end of the proximal housing component. By detaching or disconnecting the distal housing component from the proximal housing component the medicament container, e.g. a cartridge filled with the medicament, can be replaced on demand.

Furthermore, the distal housing component comprises a connecting end for detachably connecting to a complementary shaped connecting end of the proximal housing component. Typically, the connecting end of the distal housing component is provided at the proximal end of the distal housing component. The connecting end of the proximal housing component is provided at a distal end of the proximal housing component.

Typically and according to a further example one of the connecting end of the distal housing component and the connecting end of the proximal housing component comprises an insert portion configured for insertion into a complementary shaped receptacle provided on the other one of the connecting end of the distal housing component and the connecting end of the proximal housing component.

This way, an insert can be received inside the receptacle by way of a longitudinal insert motion. When reaching a final assembly configuration the insert portion is at least partially if not entirely received inside the receptacle. Here, a sidewall of the insert portion is located inside a sidewall of the receptacle. Accordingly, the insert portion and the receptacle form a nested or convoluted configuration of the distal and the proximal housing component. Here, the electronic sensor provided at the distal end of the proximal housing component and the electric identifier provided at the proximal end of the distal housing component may overlap in longitudinal and/or radial direction so as to cooperate or to interact contactlessly.

Typically, the drive mechanism provided on or in the proximal housing component is resettable. When the housing components are disconnected the drive mechanism is typically subject to a reset operation so that the injection device can be set into an initial configuration after inserting a new medicament container into the distal housing component and upon reconnecting the distal housing component with the proximal housing component.

According to a further example the electric identifier is arranged on or is fixed on one of the connecting end of the distal housing component and a proximal end of the medicament container. The electric identifier may be provided on an outside surface of the connecting end of the distal housing component or on the outside surface of a proximal end of the medicament container. With some examples the electric identifier is arranged on one of an inside surface of the connecting end of the distal housing component and an inside surface of a sidewall of the medicament container. It may be located outside a filling volume of the medicament container which is intended to be filled with the injectable medicament.

With some examples the electric identifier is embedded inside a sidewall of the distal housing component. This way, the electric identifier can be integrated non-visibly inside the structure of the housing component. With some examples, the electric identifier comprises an electrically conductive structure, e.g. a helically wound wire, which is insert molded in an injection molded sidewall of the distal housing component.

With a further example the electric sensor is arranged on or in the connecting end of the proximal housing component. It may be provided on an outside surface of a sidewall of the proximal housing component or on an inside surface of the sidewall of the proximal housing component. With some examples the electric sensor is arranged inside or is embedded inside the sidewall of the proximal housing component. Also here, the electric sensor may comprise a helically wound conductive wire embedded or integrated into the sidewall of the proximal housing component. It may be insert molded in an injection molded proximal housing component.

Generally, there exist many approaches to implement or to provide a helically wound wire of one of the electric identifier and the electric detector in or on a sidewall of at least one of distal housing component or proximal housing component. With some examples, a helically wound wire is insert molded in the sidewall, e.g. by a two- or more-component injection molding process by way of which the sidewall of the housing is formed or constituted.

With other examples, the helically wound wire is wrapped or coiled on an outside surface of a cylindrical body. Then, the cylindrical body provided with the helically wound wire is inserted into a receptacle of a complementary shaped distal or proximal housing component. The cylindrical body is fastened to the respective housing component, e.g. by using an adhesive or by a press-fit. In either way, the helically wound wire is located between an outside surface of the body and an inside surface of the sidewall of the respective housing component. With some examples, wherein the electric identifier is provided on an outside surface of the distal housing component the electric sensor is provided on an outside surface of the proximal housing component or it is located inside the sidewall of the proximal housing component. With such a configuration the electric sensor is not provided on an inside surface of the proximal housing component. With other examples and wherein the electric identifier is located on an inside surface of a sidewall of the distal housing component or wherein the electric identifier is embedded inside a sidewall of the distal housing component the electric sensor may be provided on one of an inside surface of the sidewall of the proximal housing component, on an outside surface of the sidewall of the proximal housing component or it may be located inside the sidewall of the proximal housing component.

Typically, the material of the sidewall of the proximal housing component and/or the material of the sidewall of the distal housing component is electrically insulating. The proximal housing component and/or the distal housing component may comprise an injection molded plastic material. This way, and when the proximal and distal housing components are in a final assembly configuration the material of the sidewall of the respective housing components provides electrical insulation between the electric sensor and the electric identifier.

With some examples the electric identifier is located on the medicament container. With other examples the electric identifier is located on the distal housing component, wherein the distal housing component is preassembled with the medicament container. Here, the medicament container may be non-detachably connected to the distal housing component. Typically, the distal housing component is detachably connectable and/or detachably fixable to the proximal housing component.

According to a further example the electric identifier comprises an inductive coil or a solenoid coil with a coil axis extending substantially parallel to the longitudinal direction of the housing. The coil may comprise numerous helical windings. The number of windings may specify a well-defined inductive response of the coil when excited by a magnetic or electromagnetic field, e.g. provided by the electric sensor.

The inductive coil may extend around the sidewall of the distal housing component and/or around the sidewall of the medicament container. With some examples the medicament container comprises a tubular-shaped barrel, e.g. made of a vitreous material. The inductive coil of the electric identifier may extends around the outer or inner circumference of the respective tubular shaped structure of the medicament container and/or of the distal housing component. The inductive coil may encircle or enclose the sidewall of the medicament and/or the sidewall of the distal housing component.

According to a further example the electric identifier comprises a floating coil. Here, the inductive coil is closed in itself. The electric identifier may be void of external contact pins or contact pads. This enables a rather easy and straightforward fastening or embedding of the electric identifier on or in the distal housing component and the medicament container.

With an electric identifier being void of contact pads or contact pins the electric identifier can be arranged in any arbitrary angular orientation with regard to its coil axis. This provides a rather simple and easy way of how to assemble the electric identifier on or in the distal housing component and/or medicament container.

According to a further example the inductive coil is provided on a flexible substrate configured for attachment or arrangement to a tubular-shaped sidewall of one of the distal housing component and the medicament container. The flexible substrate may comprise a bendable or pliable foil by way of which the inductive coil can be attached to at least one of the tubular-shaped sidewall of the distal housing component and the medicament container. The inductive coil may comprise a printed electric conductor attached to the flexible substrate by way of printing. Printed electrically conductive structures on a flexible substrate can be manufactured at moderate or low costs and in large quantities. This enables use of the electric identifier in a mass manufacturing environment, wherein a vast number of housing components or medicament containers can be electrically and/or electronically encoded. Such housing components or medicament containers may be configured or implemented as disposable components, i.e. components which are intended to become discarded after use.

According to a further example the flexible substrate comprises an adhesive layer for adhesively attaching the electric identifier to the sidewall of one of the distal housing component and the medicament container. The adhesive layer and the inductive coil may be provided on the same side or on opposite sides of the flexible substrate. In either way the adhesive layer may provide a durable, non-detachable connection of the electric identifier on one of the distal housing component and the medicament container.

The substrate may form or may comprise a label configured for attachment to a sidewall of the medicament container or distal housing component. The label may comprise printed information being indicative of the medicament or use of the medicament. Here, the inductive coil may be integrated into the label and the outer appearance of the medicament container or distal housing component may remain visually unaffected by the coil implementation.

According to a further example the passive electronic identifier is an unpowered electronic identifier being void of a permanent source of electric energy. This allows to reduce manufacturing costs and to save storage space for implementing the identifier on the medicament container or on the distal housing component. A passive or unpowered electronic identifier can be manufactured in large quantities at low or moderate costs. It can be implemented or configured as a disposable electronic identifier.

According to a further example the passive electronic identifier comprises a microchip and an antenna. The antenna is configured to harvest electric energy from a surrounding electromagnetic field. The coil or solenoid of the inductive identifier can be implemented as the internal; or vice versa. With some examples the passive electronic identifier is implemented as a near field communication (NFC) tag. With some examples the passive electronic identifier comprises a passive RFID tag. Furthermore, and with some examples the passive electronic identifier comprises a near field communication tag configured to communicate with the electric sensor implemented as a corresponding near field communication interface of the electronic unit.

According to a further example the electric identifier comprises a nonvolatile memory to store at least one of a medicament name, a medicament manufacturing date, a medicament expiry date, a LOT number of the medicament or a unique medicament container ID. In this way, a rather large variety of medicament related data can be stored in or by the identifier and can be captured or retrieved through a readout of the identifier.

According to a further example the nonvolatile memory of the electric or electronic identifier is at least one of erasable or writable by the electric sensor. In this way, the nonvolatile memory of the passive electronic identifier can be reconfigured. There may be provided a validation flag in the nonvolatile memory indicating if the respective electronic identifier has been subject to a read or write operation before. By providing an erasable or writable nonvolatile memory, such a read or invalidation flag can be set or reconfigured upon a first or repeated reading or readout of the nonvolatile memory by the electric sensor. Here, the electric sensor can be implemented as an inductive transmitter.

In this way, patient safety can be enhanced so as to prevent a multiple sequential use of one and the same medicament container, e.g. with the same or with different injection devices. In particular, a medicament container provided with a unique medicament container ID can be marked to be invalidated, so that an abuse of the container, e.g. a non-intended refilling of the container becomes somewhat effectless because then, such a counterfeited container will be inoperable to deactivate the interlock of the injection device.

According to a further example the electric sensor comprises an inductive coil or a solenoid coil with a coil axis extending substantially parallel to the longitudinal direction (z) of the housing. With this example and when the distal and proximal housing components are finally assembled in the final assembly configuration with the medicament container arranged inside the housing the inductive coil of the electric identifier and the conductive coil of the electric sensor are co-aligned and extend coaxially with respect to each other. This way, there may be provided a longitudinal and/or radial mechanically contactless overlap between the inductive coils of the electric identifier and the electric sensor, respectively.

This provides a well-defined inductive coupling between the respective inductive coils so that measurable characteristics of the electric identifier, such as a characterizing analogue or digital response, e.g. a characteristic resonance frequency or other inductively measurable parameters can be detected.

The inductive coil of the electric sensor may comprise numerous helical windings extending around an inner or outer circumference of a tubular-shaped sidewall of the proximal housing component. The coil of the electric sensor may be arranged on, in or inside the distal connecting end of the proximal housing component. In the final assembly configuration of the proximal and the distal housing components the inductive coil of the electric sensor may enclose or encircle the inductive coil of the electric identifier; or vice versa.

The inductive coil of the electric sensor comprises a first end provided with a first connector and further comprises a second end, opposite the first end and provided with a second connector. First and second connectors are electrically connected with the electronic unit.

With some examples, the inductive coil of the electric sensor and the inductive coil of the electric identifier overlap with regard to the longitudinal direction of the housing. The coils may be oriented and/or arranged coaxial with regard to each other when the respective housing components are mutually connected or assembled. When the inductive coils of the electric sensor and the electric identifier overlap with regard to the longitudinal direction, one the coils has a diameter smaller than the diameter of the other coil so as to enable a longitudinally nested and concentric mutual arrangement of the coils.

With other examples the inductive coil of the electric sensor and the inductive coil of the electric identifier are arranged in a non-overlapping configuration as seen in longitudinal direction. Also here, the coils may be arranged and/or oriented coaxial with regard to each other but they may comprise substantially equal diameters. Here, and when the distal housing component and the proximal housing component are in a mutual assembly configuration and when the distal housing component and the proximal housing component are connected to each other the inductive coil of the electric identifier may be located at a longitudinal offset from the inductive coil of the electric detector. With some examples, the inductive coil of the electric identifier may longitudinally adjoin the inductive coil of the electric detector while the respective coils remain electrically insulated from each other.

In this way, the radial dimensions of the inductively coupled coils of the electric identifier and of the electric detector may be somewhat equal in size, thus allowing for a rather easy and intuitive attachment to the respective housing components.

In effect, for achieving an inductive coupling it may not be necessary that the coils of the electric identifier and of the electric detector overlap as seen in longitudinal direction. Since the inductive coupling can be also achieved when the coils are arranged at a longitudinal distance from each other or when the respective inductive coils adjoin each other in the longitudinal direction there can be provided an increased degree of flexibility for arranging and attaching respective inductive coils to at least one of the distal housing component, the proximal housing component and the medicament container.

According to a further example the at least one interlock of the drive mechanism comprises an electromechanical interlock configured to mechanically engage with a first component of the injection device and with a second component of the injection device. The first component is movable relative to the second component for at least one of setting of the dose and injecting of the dose of the medicament. The at least one interlock is operable to switch between an interlocked state, in which the first component is movably locked to the second component and a release state, in which the first component is movable relative to the second component, typically for at least one of setting of the dose and dispensing or injecting of the dose.

With some examples the first component is movable in longitudinal direction relative to the second component for at least one of setting of the dose and injecting of the dose. With other examples the first component is rotatable relative to the second component for at least one of setting of the dose and injecting of the dose. With a rotatable relative displacement between the first component and the second component an axis of rotation typically extends along the longitudinal direction or axial direction of the housing of the injection device.

With some examples the first component is subject to a helical motion relative to the second component for setting and/or injecting of the dose of the medicament. At least one of the first and the second components may belong to the drive mechanism. With some examples, both, the first and the second components belong to the drive mechanism of the injection device. With other examples at least one of the first and the second component is or forms part of the housing of the injection device.

Typically and with a further example the electromechanical interlock comprises an electrically implemented actuator operably engaged with a latch. With some examples the latch is movable by the actuator along a longitudinal direction, along a radial direction and/or along a tangential direction with regards to a cylindrical or tubular geometry of the housing of the injection device. Typically, the actuator is located and/or fixed on one of the first and second components. The latch may be provided on the same component and may be mechanically engageable with the other one of the first and second component in order to block or to impede a relative movement of the first component and the second component when the interlock is in the interlocked state.

With some examples the drive mechanism of the injection device comprises a piston rod, a number sleeve, a drive sleeve or driver and at least one of a dose dial and a trigger or dose button. Typically, for setting of a dose the dose dial is rotatable relative to the housing. During dose setting the rotational movement of the dial is transferred to a respective rotational movement of the number sleeve. Here, and for impeding setting of a dose, at least one of the dose dial and the number sleeve may be rotationally and/or translationally locked by the interlock.

For dispensing or injecting of the dose the trigger or dose button has to be depressed by a user. The trigger button may be depressible relative to the dose dial, relative to the housing, relative to the number sleeve or relative to the piston rod. During dose dispensing the driver or drive sleeve is typically subject to a rotational movement that is transferred into a longitudinal and/or rotational movement of the piston rod. During dose injection the number sleeve typically rotates in a direction opposite to a direction of rotation during setting of the dose. Here, the interlock may be operable to block actuation or movement of the trigger, to block a rotation of the driver or drive sleeve, to block a rotation or movement of the piston rod and/or to block or to inhibit rotation or movement of the number sleeve.

Apart from these example further implementations of the interlock or electromechanical interlock are possible and conceivable as long as the first and second component to be mutually locked and engaged are subject to a relative movement during at least one of setting of the dose and dispensing of the dose.

According to a further example the drive mechanism comprises at least one clutch operable to switch the drive mechanism between a dose setting mode and a dose injecting or dose dispensing mode. The at least one interlock is operably engaged with the at least one clutch to selectively disable switching of the drive mechanism from one of the dose setting mode and the dose injecting mode into the other one of the dose setting mode and the dose injecting mode.

With some examples the drive mechanism comprises a first clutch and a second clutch. By way of a first clutch the number sleeve can be selectively engaged with a dose dial, a driver or drive sleeve of the drive mechanism. In this way, a rotation of the dose dial can be transferred into a respective rotation of the number sleeve during setting of the dose. During injecting of the dose the number sleeve can be decoupled and released from the dose dial. Here, the number sleeve is allowed to rotate in an opposite sense of rotation while the dose dial does not have to follow this rotation but remains rotationally locked to the housing of the injection device.

By way of a second clutch the dose dial and/or the number sleeve can be selectively engaged with a driver or drive sleeve of the drive mechanism. Furthermore, by way of the second clutch and/or by way of a third clutch there may be provided a selective engagement between a driver or drive sleeve and the piston rod so as to move the piston rod in distal direction exclusively during injecting of the dose.

Generally, the drive mechanism of the injection device can be implemented in various different ways. One example of a drive mechanism is for instance disclosed in WO 2004/078239 A1 the entirety of which being incorporated herein by reference. Another example of a drive mechanism to be implemented with the injection device is for instance disclosed in WO 2014/033195 A1, the entirety of which being incorporated herein by reference.

According to a further example the at least one interlock is controlled by a processor of the electronic unit. The processor is configured to activate the at least one interlock if one or several of the following conditions have been detected by the electronic unit. Such conditions may be: end of a dose injection, depletion of the medicament container, lapse of a predefined time interval since deactivation of the at least one interlock, removal of the medicament container from the housing, insertion of the medicament container into the housing, conducting of a manual reset of the drive mechanism or lapse of a predefined time interval since a communication link between the near field communication interface of the electronic unit and the identifier abrogated.

There may be provided any further conditions by way of which the processor is triggered to activate or to de-activate the interlock. Typically, and with any of the above conditions an automated activation of the interlock can be provided, thereby effectively preventing unintended or non-authorized use of the injection device. Any of the above mentioned conditions can be detected e.g. by at least one sensor as described further below.

According to a further example the injection device comprises the medicament container filled with the medicament. The medicament container is arranged inside the housing. Typically, the medicament container comprises a cartridge filled with the medicament. The cartridge may comprise a tubular-shaped barrel, e.g. made of a vitreous material. The barrel may be sealed towards the proximal direction by a stopper movable along the longitudinal direction of the barrel for expelling the liquid medicament through a distally located outlet of the barrel.

The distally located outlet may be sealed by a seal. The seal at the distal end of the cartridge may comprise a sealing disc, e.g. made of a rubber material and being pierceable by a double-tipped injection needle. The rubber seal may be fixed or assembled to the distal end of the tubular barrel by way of a crimped metal cap.

According to a further example the medicament container is non-removably attached to the distal housing component. The distal housing component typically comprises a tubular-shaped cartridge holder shaped and configured to accommodate a medicament cartridge therein. The cartridge holder may be provided with a snap feature in order to fix the cartridge inside the cartridge holder. In this way, there may be provided a cartridge holder cartridge pre-assembly that may be commercially distributed to end consumers or patients. With such a preassembly the electric identifier may be provided on an outside surface of the distal housing component, hence on an outside surface of the cartridge holder. It may be also provided on the sidewall of the medicament container, i.e. on the barrel of the cartridge.

According to a further example the electronic unit of the injection device comprises at least one sensor to detect or to measure movement of at least one movable component of the injection device. The movable component of the injection device may be indicative of a particular device configuration or device operation. By detecting the movement or the magnitude of the movement of the movable component the electronic unit is provided with information regarding the operational status of the injection device.

Typically and with some examples the sensor is operable to quantitatively measure the degree of movement of the at least one movable component. The at least one sensor may be operable to measure a degree of rotation and/or a degree of longitudinal movement of the at least one movable component, e.g. relative to the housing of the injection device or relative to another movable component of the drive mechanism of the injection device.

By way of the at least one sensor there may be derived data with regards to a dose size. Here, the at least one sensor is operable to provide electrical signals being indicative of a size of a dose actually set and/or dispensed or injected by the drive mechanism of the injection device. Typically, the electronic unit is provided with a memory as well as with a clock. In this way a dosing history or injection history can be automatically captured and stored in the memory of the electronic unit.

The electronic unit may be further equipped with a short range communication interface, e.g. comprising a wireless communication interface based on one of the following communication standards, Bluetooth, Bluetooth low energy (BLE), ZigBee or Wi-Fi. Typically, the short range communication interface of the electronic unit is operable to set up a communication link with a corresponding short range communication interface of an external electronic device. The external electronic device may be implemented as a portable electronic device, such as a smartphone, a smart watch or a tablet computer.

Especially by way of the optional short range communication interface and by way of a communication link with the external electronic device dispensing- and hence injection-related data, such as an injection history can be synchronized with the external electronic device and can be transmitted to medical service providers for further data analysis. In this way, repeated use of the injection device can be precisely tracked and measured and the user's compliance with a given prescription schedule can be monitored.

According to a further aspect the present disclosure relates to a kit of injection devices, in particular to an injection device of a first type and at least to an injection device of a further type, e.g. a second type. Injection devices of the first type and of the second type distinguish by their medicament or medicament container. The medicament container of the first type may distinguish from a medicament container of the second type by its size, the type of medicament, the pharmaceutically active substance and/or by the concentration of the pharmaceutically active substance.

The medicament container of the first type is exclusively configured for use with a drive mechanism of a first type. The medicament container of the second type is exclusively configured for use with a drive mechanism of a second type. Unintended cross use, i.e. use of a medicament container of the first type with an injection mechanism of the second type should be avoided.

For this, the medicament container of the first type is equipped and/or associated with an electric identifier of the first type. The medicament container of the second type is equipped and/or associated with an electric identifier of the second type. By way of the electric identifier the respective type of the medicament container can be detected. Here, the identifier provides a kind of a coding. The identifier of the first type represents a first coding. The identifier of the second type represents a second coding. The identifiers of different types are detectable and/or readable by the electric sensor.

Electric signals generated by the electric sensor when interacting with the electric identifier are provided and transmitted to the electronic unit, which is capable to identify the electric identifier and the associated housing component or medicament container on the basis of such electric signals, i.e. on the basis of so-called detection signals. The electronic unit, in particular a processor of the electronic unit is further configured to validate the detection signals and to at least temporarily deactivate or re-activate the at least one interlock on the basis of the validation of the detection signals obtained from the electric sensor through interaction with the electric identifier.

The electric identifiers may represent an electronic coding or a coding that is electronically detectable. Such identifiers or codings enable use of mechanically unencoded housing components for the injection devices of the first and the second type. Even though a distal housing component equipped with a medicament container of the first type is assembled with a proximal housing component of a second type and equipped with a second type of an injection device such a combination will not be operable to abrogate or to deactivate the interlock of the drive mechanism. Setting and/or injecting of a dose will be effectively impeded.

According to another aspect the present disclosure relates to a method of unlocking operation of an injection device as described above for at least one of setting or injecting of a dose of a medicament. The injection device comprises a housing of elongated shape and extending along a longitudinal direction. The housing comprises a distal housing component and a proximal housing component. The distal housing component is configured to accommodate or to receive a medicament container as described above. The proximal housing component is configured to accommodate or to support a drive mechanism to operably engage with the medicament container for expelling or withdrawing of the dose of the medicament from the medicament container. Typically, the injection device comprises a drive mechanism arranged inside the proximal housing component.

The injection device further comprises an electronic unit coupled to the drive mechanism. The injection device also comprises an electric sensor provided at or near a distal end of the proximal housing component. The electric sensor is electrically connected to the electronic unit and is configured to detect an electric identifier provided on or in one of the medicament container and the distal housing component. The method of unlocking operation of the injection device comprises the step of detecting at least one of a presence and a type of an electric identifier by the electric sensor. This detection may take place before, during or after mutual assembly of the distal and proximal housing components with the medicament container located inside the distal housing component.

After contactlessly detecting the electric identifier by the electric sensor there is generated at least one detection signal, e.g. by the electric sensor. The at least one detection signal is indicative of the electric identifier. The at least one detection signal is then transmitted to the electronic unit. The electronic unit processes or validates the detection signal and at least temporarily deactivates or reactivates the at least one interlock on the basis of the validation of the detection signal.

Generally, the method of unlocking operation is to be conducted by an injection device as described above being equipped with an interlock configured to disable at least one of setting of the dose and dispensing of the dose. Insofar, all features, effects and benefits as described above in connection with the injection device equally apply to the method of unlocking operation of the injection device.

According to a further example the method comprises the step of validating the electric identifier detection signals obtained from the electric identifier by comparing the detection signals obtained from the electric identifier with predefined data related to the injection device. Such predefined data may be stored in a local storage of the electronic unit. In this way it can be determined or asserted if the electric identifier matches with predefined data related to the injection device and if the medicament container is usable or is intended for use with the injection device.

If the acquired or captured electric identifier-related data does not match with predefined data related to the injection device the drive mechanism is either actively locked or remains locked by the interlock. Then, the injection device is effectively inoperable to set a dose of the medicament and/or to inject a dose of the medicament. With further examples the method and the step of validating the identifier may include to modify the identifier. This may particularly apply with a digital implementation of the electric identifier and by a digital encoding thereof.

Here, and with some examples the electronic unit of the injection device may be operable to read out the electronic storage of the passive electronic identifier. Upon such a readout activity a respective readout flag of the electronic identifier may be set so as to mark the electronic identifier as being currently in use or as having been used. A repeated use of the passive electronic identifier and/or of the medicament container associated with the identifier may be then effectively impeded or blocked, e.g. to prevent or to impede an unintended and/or unauthorized use of the medicament container. Insofar, capturing or reading of the identifier may include an evaluation or approval of the status of the readout flag provided by the electronic storage of the electronic identifier.

Alternatively and with further examples it is even conceivable that upon readout of the identifier provided on or associated with the medicament container by the electric sensor there is invoked a database request with an external database, e.g. via a network connection. In the database the medicament container currently in use may be marked to be ready for use or of not being suitable for use. With such a database request additional information may be gathered if a particular medicament container can be used or should not be used or cannot be used with the injection device.

According to another aspect there is provided a label for attachment to at least one of a housing of an injection device or to a medicament container configured for insertion into the housing of the injection device. The label comprises a flexible substrate provided with a conductor strip. The flexible substrate is wrappable or coilable into a wrapped or coiled configuration, such that the conductor strip forms an inductive identifier. In the wrapped or coiled configuration the conductor strip comprises a helical structure with at least one or numerous helical windings effectively providing an inductive coil.

With some examples the conductor strip is coated or printed on a first side of the flexible substrate. A second side of the flexible substrate is provided with an adhesive, e.g. with an adhesive layer for fixing the substrate to a sidewall of at least one of a distal housing component, a proximal housing component or a medicament container of an injection device. With other examples the adhesive and/or the adhesive layer is provided on the first side. Here, the adhesive and the conductor strip are provided on the same side of the flexible substrate.

With some examples the conductor strip comprises a first elongated conductor strip portion and a second elongated conductor strip portion, wherein the first elongated conductor strip portion extends at a predefined angle relative to the elongation of the second elongated conductor strip portion. With some examples the second elongated conductor strip portion extends substantially parallel or coaxial with an axis of symmetry of the wrapped or coiled flexible substrate. Here, the second elongated conductor strip extends parallel to a coil axis of the coiled or wrapped flexible substrate. The first elongated conductor strip may then form the helically wound portion of the conductor strip.

With some examples the conductor strip comprises a first longitudinal end provided with a first contact zone and further comprises a second longitudinal end provided with a second contact zone. The first and second contact zones comprise a well-defined size and/or geometry of e.g. quadratic, rectangular, round or oval shape. They comprise a diameter or cross-section that is larger compared to a diameter or cross-section of the elongated conductor strip.

With further examples and when the flexible substrate is in a coiled configuration forming at least one or numerous wraps or windings, the first contact zone and the second contact zone may at least partially overlap as seen in a radial direction with regards to the coil axis.

With further examples, the substrate is electrically insulating. In the coiled or wrapped configuration the substrate is located radially between the at least partially overlapping first and second contact zones. This way, the first and second contact zones and the electrically insulating material located therebetween effectively form a capacitor.

The capacitor as provided by overlapping contact zones together with the coiled or wrapped configuration of the conductor strip located therebetween forms a LC circuit configured to characterize the respective label. The LC circuit itself may be characterized by the number of windings of the conductor strip and/or by the capacitance of the capacitor as formed by the mutually overlapping contact zones.

By way of modifying the number of windings as well as by way of modifying the capacitance of the capacitor there can be provided a large number of differently scaled LC circuits, each of which featuring a well-defined resonance frequency. In this way, there can be provided a label configured for attachment to a tubular body of a housing of an injection device or to a tubular barrel of a medicament container.

Generally, the label may be also provided with a printed label portion thus providing readable or visible information with regard to the injection device or medicament.

The label in its coiled configuration provides an inductive coil that can be inductively coupled with an electric detector, e.g. equipped with a suitable inductive detector or reader, such as a reader coil. The substrate with a printed or coated conductive strip is suitable for a rather cost efficient mass manufacturing process and is hence attractive for labeling components of an injection device in an easy and cost-efficient way.

The electronic label can be void of any logic electronic components or semiconducting materials. This enables a rather ecologically friendly implementation of an electrically and/or inductively readable label.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, an example of an injection device with an electronic unit will be described in greater detail by making reference to the drawings, in which:

FIG. 1 schematically illustrates a longitudinal cross-section of one example of an injection device,

FIG. 2 schematically shows the injection device according to FIG. 1 with the distal housing component disconnected from the proximal housing component,

FIG. 3 shows a longitudinal cross-section through an example of a drive mechanism implemented with the injection device,

FIG. 4 exemplary shows the interaction between the electronic unit of the injection device with an external electronic device,

FIG. 5 is a block diagram of the injection system comprising an injection device and an external electronic device,

FIG. 6 shows a distal housing component provided with an identifier,

FIG. 7 shows a distal housing component and a medicament container provided with the identifier,

FIG. 8 shows an example of an electric identifier,

FIG. 9 shows another example of an electric identifier,

FIG. 10 shows another example of an electric identifier,

FIG. 11 schematically illustrates implementation of an electric sensor,

FIG. 12 is indicative of two diagrams showing various signals of the electric sensor,

FIG. 13 shows a flowchart of a method of unlocking operation of the injection device,

FIG. 14 is illustrative of a proximal end of the distal housing component,

FIG. 15 is illustrative of a distal end of the proximal housing component,

FIG. 16 shows a longitudinal cross-section through the housing components of FIGS. 14 and 15 when mutually assembled,

FIG. 17 shows an example of a label configured for fixing to one of the distal housing component and the proximal housing component and provided with a coil,

FIG. 18 shows another example of a label configured for fixing to one of the distal housing component and the proximal housing component and provided with a coil, and

FIG. 19 shows the label according to FIG. 18 in a wrapped or coiled configuration.

### DETAILED DESCRIPTION

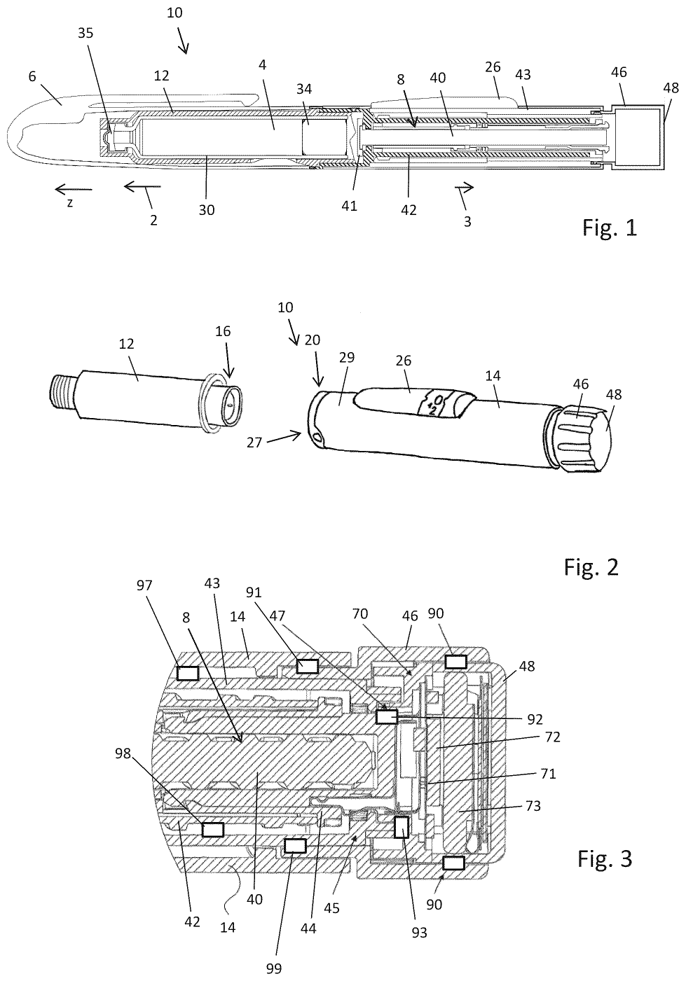

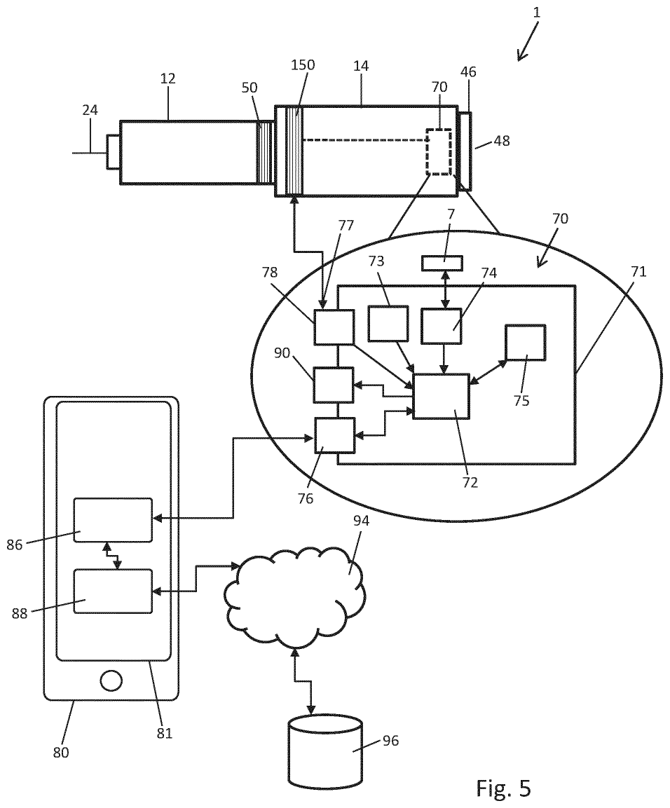

In FIGS. 1-3, 6 and 7 details of an example of an injection device 10 implemented as a pen-type injector are schematically illustrated. The injection device 10 may be part of an injection system or drug delivery system 1 as illustrated in FIG. 5. The injection device 10 comprises a housing 11. The housing 11 may be of elongated or tubular shape. The housing 11 extends in a longitudinal or axial direction (z). Towards the distal direction 2 the injection device 10 comprises a protective cap 6 covering a distal housing component 12. The housing 11 further comprises a proximal housing component 14 detachably connectable with the distal housing component 12. As becomes apparent from FIGS. 1, 2 and 6 the proximal housing component 14 comprises a connecting end 20 towards the distal direction 2.

The connecting end 20 of the proximal housing component 14 comprises a receptacle 27 configured to receive a complementary shaped proximal connecting end 16 of the distal housing component 12. As illustrated in FIGS. 2 and 6, the proximal connecting end 16 of the distal housing component 12 comprises an insert portion 17 configured for connection with the distal connecting end 20 of the proximal housing component 14. For this, the proximal housing component 14 comprises the receptacle 27 at the distal connecting end 20 configured and shaped to receive the insert portion 17 therein.

On an outside surface of the insert portion 17 there is provided at least one fastening element 19, e.g. implemented as a protrusion to mate or to engage with a complementary-shaped recess on an inside surface of a sidewall of the receptacle 27. The insert portion 17 and the receptacle 27 may comprise mutually corresponding or complementary shaped fastening elements, so as to form a snap fit engagement, a bayonet joint or a threaded joint.

The insert portion 17 is delimited in longitudinal direction by a radially outwardly extending flange 18. When the housing components are fully assembled the flange 18 is in proximal longitudinal abutment with a distal end face of a sidewall or of a distal end of the proximal housing component 14.

The distal housing component 12 is implemented as a cartridge holder. It comprises a tubular-shaped sidewall 13 configured and sized to receive a medicament container 30 as illustrated in FIG. 7. The cartridge holder as shown in FIG. 7 comprises at least one aperture 15 in its tubular-shaped sidewall 13. Towards the distal end the tubular-shaped sidewall 13 radially narrows and forms a shoulder portion adjoining into a distal connecting end 20. The medicament container 30 is typically filled with an injectable medicament 4, e.g. provided or stored in liquid form inside the medicament container 30.

The connecting end 20 comprises a mount 21 for a needle assembly 23. The needle assembly 23 comprises a needle hub 24 configured for detachable or releasable engagement with the mount 21 and further comprising an injection needle 25. The injection needle 25 is typically implemented as a double-tipped injection needle. When correctly assembled on the mount 21 a proximal tipped end of the injection needle 25 reaches through a distal through opening 22 at the connecting and 20 and pierces a seal 35 provided at the distal end of the medicament container 30 located inside the cartridge holder.

The seal 35 may be implemented as a rubber disc and may serve as a pierceable septum. It may be mechanically fixed to the distal end of the barrel 31 of the medicament container 30 by way of a crimped metal cap 36. Towards the proximal end the tubular shaped barrel 31 of the container 30 is sealed by a stopper 34, e.g. implemented as a rubber stopper. The piston or stopper 34 is movably disposed inside the barrel 31. It may be displaceable towards the distal direction 2, hence towards the dispensing end of the injection device 10 through the action of a piston rod 40 of a drive mechanism 8 provided and assembled inside the proximal housing component 14.

The drive mechanism 8 can be implemented in many different ways. Insofar the present disclosure is by no way limiting to the presently illustrated example of a particular drive mechanism 8. With the presently illustrated example the drive mechanism 8 comprises a piston rod 40 equipped with a pressure piece 41 at its distal end. The pressure piece 41 is configured to apply distally directed pressure onto the stopper 34 of the medicament container 30. The drive mechanism or the injection device further comprises a housing insert 42, which serves as a mount for numerous movable components of the drive mechanism 8.

Typically, the drive mechanism 8 comprises a number sleeve 43 provided with a consecutive sequence of numbers on an outside facing sidewall. These numbers are visible through a window 26 or through a respective aperture in the sidewall 29 of the proximal housing component 14. The drive mechanism 8 further comprises a driver 44, which may be implemented as a drive sleeve. For setting of a dose the number sleeve 43 is typically subject to a rotation relative to the housing 11 along a first sense of rotation. During dispensing or injecting of the dose the number sleeve 43 is subject to a rotation along a second and hence opposite sense of rotation.

The driver 44 or drive sleeve can be permanently or non-permanently operably engaged with the piston rod 40. At least during dispensing of a dose the driver 44 is in a force- or torque-transmitting engagement with the piston rod so as to urge the piston rod 40 in distal direction 2 for dose expelling or dose injecting. With some implementations of the drive mechanism 8, the driver 44 is subject to a rotation along the second sense of rotation during dose dispensing. With some examples the driver 44 is subject to a rotation along the first sense of rotation during and/or for setting of the dose. With some examples the driver 44 is rotationally and/or axially fixed relative to the housing 11 during setting of the dose but is movable during dispensing or injecting of the dose.

The drive mechanism 8 further comprises a dose dial 46, e.g. provided at the proximal end of the proximal housing component 14. The dose dial 46 provides a user grippable or user operable tool for setting of the dose. Typically, the dose dial 46 is rotatable by a user relative to the housing 11 for setting of the dose. The drive mechanism 8 further comprises a trigger 48, e.g. implemented as a dose button depressible by a user in distal direction 2 for initiating and/or for controlling the process of dose dispensing or dose injection.

The trigger 48 and the dose dial 46 may be provided on a common part. They may be integrally formed. They may be both located on a so-called dial extension of the injection device 10, which is subject to a helical motion relative to the housing 11 during setting of the dose and which is subject to a longitudinal distally directed movement during and/or for dispensing or injecting of the dose. Typically, the trigger 48 is provided at a proximal end of the dose dial 46. It may form or constitute a proximal end face of the housing 11.

In the example of FIG. 3 there are indicated numerous possible implementations of an interlock 90, 91, 92, 93, 97, 98, 99. Here, the interlock 90 is provided as an interlock between the trigger 48 and the dose dial 46. With an activated interlock 90 a mutual displacement of the trigger 48 relative to the dose dial 46 can be effectively blocked. When the trigger 48 should be non-rotationally fastened to the housing 11 or to the dial extension an activated interlock 90 may also block a rotation of the dose dial 46 relative to the trigger 48 and hence relative to the housing 11. In this way, dispensing of a dose and/or dialing or setting of a dose can be effectively blocked or deactivated by activation of the interlock 90.

The interlock 91 is in mechanical connection with the proximal housing 14 and with at least one of the dose dial 46, the trigger 48 and the dial extension. Activation of the interlock 91 serves to block or to impede at least one of a rotation and a longitudinal sliding movement of the dose dial 46 relative to the housing 14. With an activated interlock 91 setting of a dose and/or dispensing of the dose can be effectively blocked.

The drive mechanism 8 may further comprise at least a first clutch 45 and a second clutch 47. By way of the clutches 45, 47 the drive mechanism 8 is switchable between a dose setting mode and a dose injecting mode. Depending on the specific implementation of the drive mechanism 8, the clutches 45, 47 are operable to mutually engage any two of the following components: piston rod 40, housing insert 42, housing 11, number sleeve 43, driver 44, dose dial 46 and/or trigger 48. Details of one example of a drive mechanism are further described in document WO 2014/033195 A1. The clutch 45 may be provided with a further interlock 93 and the clutch 47 may be provided with the interlock 92. The interlock 92 may be operably engaged with the clutch 47. Likewise, the interlock 93 may be operably engaged with the clutch 45.

With an activated interlock 92, 93 operation of the respective clutch 47, 45 may be blocked so as to impede a switching of the drive mechanism 8 from the dose setting mode into the dose injecting mode and/or vice versa, namely from the dose injecting mode into the dose setting mode.

With a further example as illustrated in FIG. 3 the number sleeve 43 is selectively engageable with the proximal housing component 14 via an interlock 97. With a further example the number sleeve 43 is selectively engageable with the housing insert 42 via an interlock 98. With a further example as illustrated in FIG. 3 the number sleeve 43 is selectively engageable with at least one of the dose dial 46 and the trigger 48 via another interlock 99.

By way of the interlock 97 at least one of a rotation and a longitudinal movement of the number sleeve 43 relative to the proximal housing component 14 can be blocked or impeded. By way of the interlock 98 at least one of a rotation and a longitudinal movement of the number sleeve 43 relative to the housing insert 42 can be selectively blocked or impeded. Since the number sleeve 43 is subject to at least one of a rotational, helical or longitudinal sliding movement relative to the proximal housing component 14 during setting of the dose and injecting of the dose a respective blocking of a movement of the number sleeve 43 serves to block or to impede at least one of a dose setting and a dose injecting action.

By way of the interlock 99 a movement, e.g. a rotational and/or longitudinal movement of the dose dial 46 and/or of the trigger 48 can be blocked relative to the number sleeve 43. This way and with typical implementations of the drive mechanism 8, at least injecting of a dose can be effectively impeded or prevented when the interlock 99 is activated.

With typical implementations the interlocks 90, 91, 92, 93, 97, 98, 99 each comprise an electromechanical actuator and at least one of a latch and a clamp. This way, the latch or clamp is movable between a locking configuration and a release configuration by the actuator, wherein operation of the actuator is controllable by electrical signals, e.g. obtained from or generated by a processor 72 the function of which will be described below. Typically, the latch provided on a first component of the injection device 10 is configured to engage with a second component of the injection device in a form fitting manner.

With other examples and wherein the interlock comprises a movable clamp, the clamp provided on a first component of the injection device is configured to engage with a second component of the injection device in a friction fitting manner. In either way and by activation of the interlock 90, 91, 92, 93, 97, 98, 99 there can be either provided at least one of a form fitting engagement and a frictional engagement between the first and the second component of the injection device so as to block or to impede at least one of setting of a dose and dispensing of a dose.

The actuator may comprise at least one of an electromagnet and an electromechanical drive controllable by the processor 72. By way of the actuator the latch, e.g. implemented as a movable or elastically deformable bending arm, hook or clamp is movable between a locking position, in which the interlock 90, 91, 92, 93, 97, 98, 99 is activated, and a release position, in which the interlock 90, 91, 92, 93, 97, 98, 99 is deactivated. In the deactivated configuration the interlock 90, and 91, 92, 93, 97, 98, 99 allows and supports a movement of a first component relative to a second component of the drive mechanism so as to enable at least one of setting of a dose and injecting of the dose.

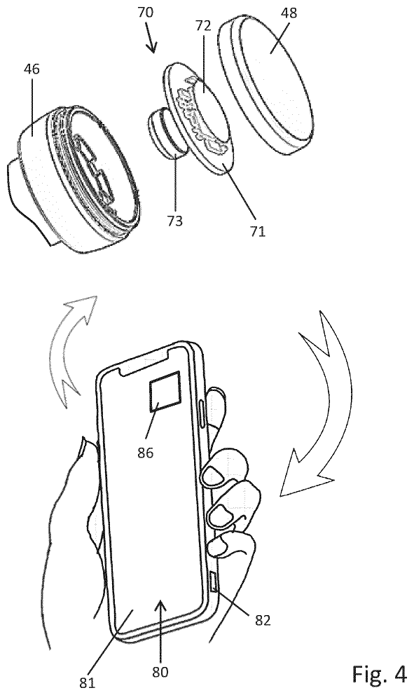

The injection device 10 is further provided with an electronic unit 70. With the present example the electronic unit 70 is embedded inside the drug delivery device 10. It is arranged at or near the proximal end of the housing 11. It may be located or arranged inside a dial extension of the injection device 10. With the example as illustrated in FIGS. 3 and 4 the electronic unit 70 is arranged inside a hollow space of the cylindrically-shaped dose dial 46, which is covered and/or closed towards the proximal direction 3 by the trigger 48. Here, the trigger 48 provides an end cap forming a proximal closure of the hollow dose dial 46. With some examples, the trigger 48 and the dose dial 46 are integrally formed.

The electronic unit 70 comprises a printed circuit board 71 provided with a processor 72. The electronic unit 70 is further provided with a source of electric energy, typically implemented as an electric battery 73. As illustrated, the processor 72 and the battery 73 may be provided on opposite sides of the printed circuit board 71.

The electronic unit 70 is schematically illustrated in the block diagram of FIG. 5. The electronic unit 70 further comprises an interface 78 electrically connected to an electric sensor 150 provided at or near a distal end of the proximal housing component 14. The electric sensor 150 may be stationary attached and/or fixed on or in a sidewall of the tubular shaped proximal housing component 14. With some examples, wherein the electronic unit 70 is movably disposed in the proximal housing component 14 or is connected to a dial extension movable relative to the proximal housing component there may be provided a sliding contact 77 by way of which the interface 78 remains in electrical contact with the electric sensor 150. The interface 78 may be implemented as a near field communication (NFC) interface.

The electronic unit 70 may further comprise a communication interface, e.g. in form of a short range communication interface 76, particularly configured to communicate with a corresponding short range communication interface 86 of an external electronic device 80.

The processor 72 is connected to the interlock 90 and is operable to control operation of the interlock 90. The interlock 90 may be implemented as an electromechanical interlock. It may comprise a latch or any other type of a mechanically movable component so as to block or to impede operation of the drive mechanism 8.

Typically, the latch or the movable component of the interlock 90 is movable through and by an actuator that is electronically controllable by the processor 72. Optionally, the electronic unit 70 is provided with a sensor 74 operable to detect or to measure movement of at least one movable component 7 of the drive mechanism 8. The movable component 7 may be represented by any of the above-mentioned movable components, e.g. the piston rod 40, the number sleeve 43, the driver 44, the trigger 48, the dose dial 46 and/or any of the clutches 45, 47. By way of the sensor 74, which may be implemented as one of an optical sensor, a capacitive sensor, an inductive sensor, an optical sensor or as an acoustic sensor, operation of the drive mechanism 8 and hence operation of the injection device 10 can be supervised and monitored.

By way of the sensor 74 information regarding the size of the dose currently set, dialed or dispensed can be obtained and can be stored in a local storage 75. Data repeatedly captured or obtained during repeated and subsequent use of the injection device 10 can be stored in the storage 75. Such injection-related data stored in the storage 75 can be synchronized by the short range communication interface 76 with the external electronic device 80 and/or with an external database 96, e.g. hosted or provided by a healthcare provider.

Typically, the external electronic device 80 is implemented as a smart phone, as a smart watch or as a tablet computer. It may comprise a wide range communication interface 88 to establish a communication link to the external database 96 by a communication network 94. Typically, the external electronic device 80 is a mobile device or wearable device. It comprises a display 81 and/or a speaker 82 to communicate with a user.

Moreover, one of the distal housing component 12 and the medicament container 30 is provided with an electric identifier 50. The electric identifier 50 is detectable or readable by the electric sensor 150 when the distal housing component 12 is connected or assembled with the proximal housing component 14. Then, the electric identifier 50 and the electric sensor 150 may spatially overlap in a contactless manner. With some examples the electric identifier 50 is provided on the insert portion 17 of the distal housing component 12 as illustrated in FIG. 7. Here, the electric sensor 150 is provided on or in the sidewall 29 of the proximal housing component 14. It may adjoin the distal end of the proximal housing component 14 and may be arranged on or inside the receptacle 27. So when the distal housing component 12 and the proximal housing component 14 are mutually assembled as e.g. illustrated in FIG. 1 the electric identifier 50 may be enclosed by the electric sensor 150. The electric identifier 50 and the electric sensor 150 may be arranged in an interleaved or convoluted way.

With the example of FIG. 6, the electric identifier 50 is located on the sidewall 13 of the distal housing component 12. It may be located on an outside or inside surface of the insert portion 17. With the example of FIG. 7, the electric identifier 50 is located on an outside or inside portion of the sidewall 32 of the barrel 31 of the medicament container 30. Typically, the electric identifier 50 is located at or near a proximal end of the sidewall 32 of the barrel 31. When the medicament container 30 is assembled inside the distal housing component 12, the electric identifier 50 is located in the region of the insert portion 17.

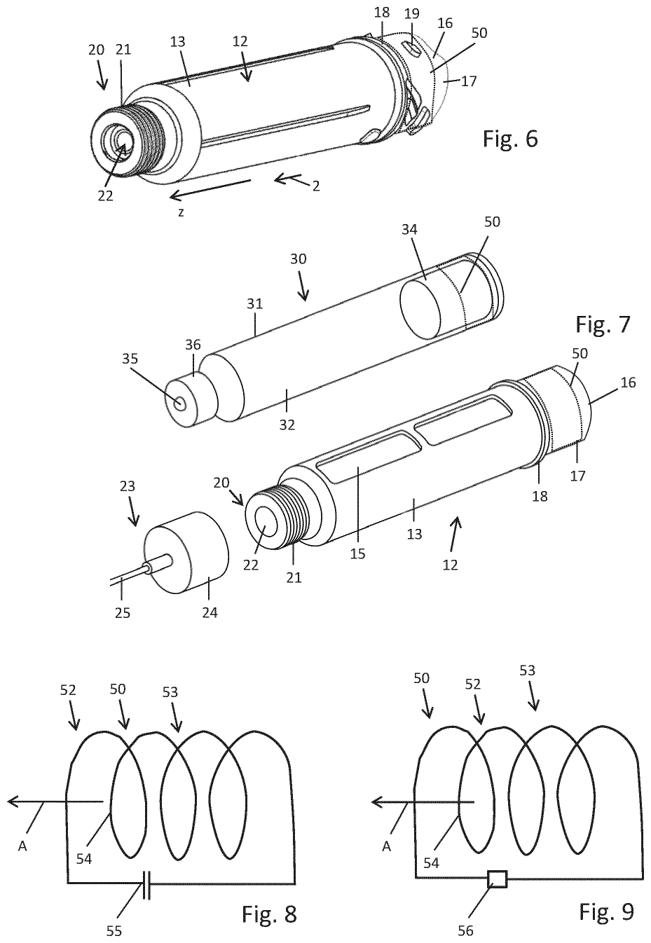

As indicated in FIGS. 8 and 9 The electric identifier 50 comprises an inductive identifier 52 comprising a coil 54 or a solenoid coil with a coil axis A extending substantially parallel to the longitudinal direction (z) of the housing 11. The inductive identifier 52 may comprise a floating coil 53, i.e. a coil which is closed in itself. The electric identifier 50 comprises a coil 54 with numerous windings 58. With the example of FIG. 8, the coil 54 is electrically connected to a capacitor 55. This way, the electric identifier provides a LC circuit with a well-defined, e.g, tunable resonance frequency. The resonance frequency of the LC circuit can be arbitrarily changed by choosing a capacitor of appropriate size and/or by making use of a coil 54 with a respective number of windings 58. In effect, the resonance frequency of the LC circuit can be changed by changing the number of windings 58 of the coil 54 by changing the capacitance of the capacitor 55.

The resonance frequency of the LC circuit may be indicative of the type of medicament 4, the size of the medicament container 30 or it may be indicative of the distal housing component 12 provided with the respective electric identifier 50.

Likewise, and as illustrated in FIG. 11, the electric sensor 150 is implemented as an inductive sensor 152. It may comprise a conductive coil 154. The conductive coil 154 may comprise a helical coil structure and may comprise a coil axis B extending substantially parallel to the elongation of the housing 11. The electric sensor 150, in particular the helical coil 154 comprises a first connector 151 and a second connector 153 at opposite ends of the helical coil 154. The coil 154 comprises several helical windings 158. The connectors 151, 153 are separately connected to the interface 78 of the electronic unit 70. They may form or belong to a sliding contact 77, in particular when the electronic unit 70 or its printed circuit board 71 should be movably disposed inside the housing 11.

With some examples the connectors 151, 153 comprise electrically conducting strips or conducting paths extending longitudinally along, inside and/or on a component of the injection device 10. With some examples, at least one of the connectors 151, 153 extends on, along or inside at least one of the sidewall 29 of the proximal housing component 14, a sidewall of the housing insert 42, a sidewall of the number sleeve 43 or any other stationary or movable component of the injection device 10. With some examples, wherein the connectors 151, 153 are arranged on, along or inside a movable component of the drive mechanism 8 there is typically also provided a sliding contact 77, by way of which electrical signals to and from the electric sensor 150 can be transmitted to and from the electronic unit 70 and/or its processor 72. With some examples and for detecting and/or characterizing the electric identifier 50 the electric sensor 150 may be designed to transmit at least one of a broadband (chirp) or frequency ramp (sweep) signal to the electric identifier 50 and to evaluate the response or load to the electric sensor 150 inductively coupled to the electric identifier 50.

Transmission or excitation of the electric identifier 50 with a sweep signal is illustrated in FIG. 12. Here, the upper diagram 200 schematically illustrates a frequency sweep signal over time. In the corresponding diagram 202, reflecting the amplitude R of a response signal over the applied frequency there can be monitored a sharp peak at the resonance frequency wo. A similar or analog characteristic response from the electric identifier may be obtained when applying a broadband frequency signal by the electric sensor.

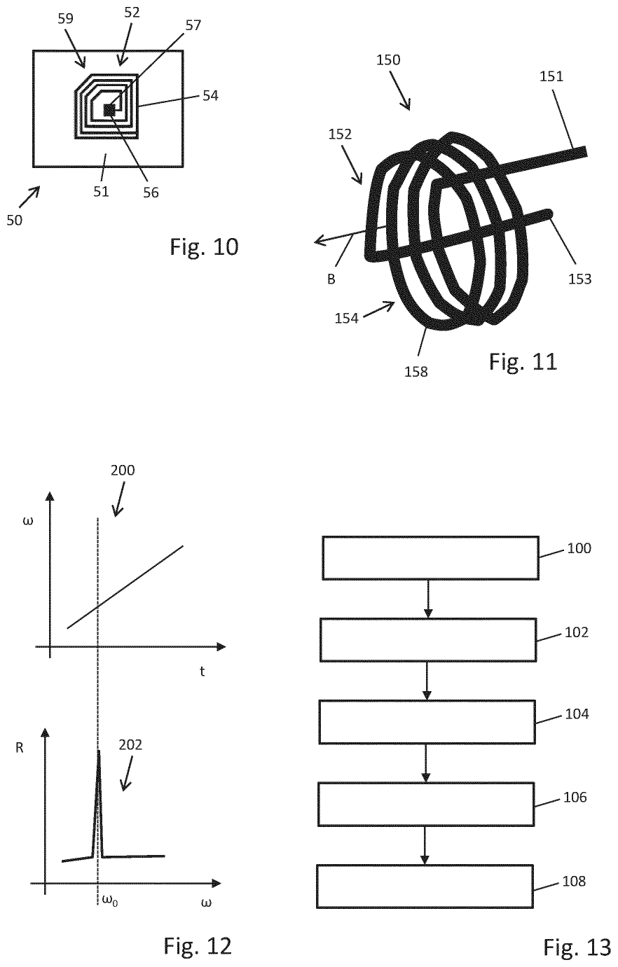

As further illustrated in FIG. 10, the electric identifier 50, e.g. an analog LC circuit or a digitally implemented microchip 56 provided with a coil 54 can be provided or printed on a planar substrate 51. The planar substrate 51 may comprise a foil on which the electrically conductive structures are printed. With some examples, the substrate 51 is provided with an adhesive layer or with an adhesive substance, e.g. on a side opposite to the coil 54. By way of the adhesive or adhesive layer, the substrate 51 and hence the entire electric identifier can be e.g. non-detachably connected to the distal housing component 12 and/or to the barrel 31 of the container 30. With some examples, the conductive structure of the inductive identifier 52 may be even embedded or insert molded in the sidewall 13 of the distal housing component 12.

With some examples, the electric sensor 150 and hence the inductive sensor 152 may be also provided on a planar substrate 51, e.g. implemented as a pliable or flexible foil, by way of which the electric sensor 150 can be assembled and/or fixed on or inside the proximal housing component 14.

With the example of FIG. 9, the inductive identifier 52 is provided with a microchip 56. Here, the microchip 56 may comprise a microprocessor and an embedded local memory 57 as illustrated in FIG. 10. Here, the coil 54 may act or behave as an antenna for RF communication with the electric detector 150. Here, the electric detector 150 may provide an inductive reader. The inductive identifier 52 provided with the microchip 56 provides a digital encoding of a medicament container 30 or distal housing component 12. Here, the electric detector 150 may transmit a well-defined signal with a well-defined carrier frequency to power up the microchip 56. In return and by harvesting of electromagnetic energy from the electromagnetic field as provided by the electric detector 150 the microchip 56 is operable to modulate a respective coil signal which modulation is detectable by the electric sensor 150.

The microchip 56 is configured to modulate a coil signal with digital information. This way, specific data related to the medicament, such as a medicament name, a medicament manufacturing date, a medicament expiry date, a LOT number of the medicament or a unique medicament container ID can be retrieved from the electric identifier 50 and can be provided to the processor 72 of the electronic unit 70.

When implemented as a passive electronic digital identifier 50 as illustrated in FIG. 10 the identifier 50 may comprise a near field communication tag 59 provided with an antenna 54 and with a microchip 56. The microchip 56 may comprise a local storage or memory 57, e.g. to store medicament related information and/or or to store medicament container related information. The memory 57 can be implemented as a read-only memory or a re-writable memory.

In either way, the identifier 50 may comprise a planar substrate 51 for attaching the identifier 52 a sidewall 13 of the distal housing component 12 or to a sidewall of the barrel 31 of the medicament container 30. The planar substrate 51 may comprise a pliable or deformable sheet structure with an adhesive layer to adhesively attached the substrate 51 and hence the identifier 50 to the housing component 12 or to the medicament container 30.

The external electronic device 80 may be wirelessly paired to the electronic unit 70. Upon successful pairing a respective confirmation may be provided to a user, e.g. via the display 81 one answers order speaker 82 of the external electronic device 80. Upon transferring of data or information of the identifier 50 between the electronic unit 70 and the external electronic device 80 a visual and/or audible confirmation may be provided to the user, e.g. by the electronic unit 70 and/or by the external electronic device 80.

If approval or processing of the data or information of the electric identifier 50 reveals that the medicament container 30 is intended for use with the injection device 1, the processor 72 is operable to deactivate the interlock 90, thereby allowing to set and/or to dispense or inject a dose of the medicament.

The processor 72 and/or the at least one interlock 90, 91, 92, 93 may be implemented in various different ways. With one approach, the interlock 90 or locking mechanism may be operable to lock setting of a dose, to lock dispensing of a dose order to lock both, setting and dispensing of a dose.

The interlock 90 and/or the processor 72 may be further configured to implement an automatic re-locking, e.g. after termination of each dispensing or in injecting procedure. Moreover and based on signals obtainable from the sensor 74 the processor 72 may be provided with information being indicative of the remaining filling level of the medicament container 30. In this way and when the sensor 74 provides respective sensor signals that the medicament container 30 is substantially depleted or empty the processor 72 may invoke activation of the interlock 92. Removal of the medicament container 30 or insertion of the medicament container 30 as well as disconnection or reconnection of the housing components 12, 14 may trigger an automated locking or disabling of the function of the drive mechanism for setting and/or for dispensing of a dose.

Information provided by the electric identifier 50 and detectable, capturable or readable by the electric sensor 150 may also provide a counterfeit protection. Here, a unique code needs to be verified, either by the electronic unit 70 and/or by a data exchange or data approval with the external database 96.

In a typical scenario of use as illustrated in FIG. 13, in a first step 100 a user may arrange the medicament container 30 provided with the identifier 50 inside the distal housing component 12. Thereafter and in step 102, the distal housing component 12 and the proximal housing component 14 are become connected, thereby bringing the electric identifier 50 provided on one of the medicament container 30 and the distal housing component 12 in an overlapping or nested arrangement with the electric sensor 150 provided at or near the distal connecting end 20 of the proximal housing component 14. Here, the coil axis A of the inductive identifier 52 may be arranged coaxially to the coil axis B of the inductive detector 152.

In the nested or spatially overlapping arrangement of the coils 54, 154, the respective coils 54, 154 remain out of mechanical contact. They are and remain electrically insulated from each other.

In the following step 104, the processor 72 may trigger a read out routine, e.g. by generating at least one or a sequence of reading signals and by transmitting the reading signals via the inductive detector 152 to the electric identifier 50. In response, and as described above, e.g. in connection with FIG. 12, the electric identifier 50 comprising a characteristic and hence well-defined resonance frequency is operable to generate a respective response signal detectable by the inductive detector 152. The inductive coupling between the mutually corresponding inductive identifier 52 and the inductive detector 152 leads to the generation of at least one detection signal by the electric sensor 150. Such detection signals are transmitted to the electronic unit 70 via the interface 78.

This way, and on the basis of detection signals obtainable from the inductive detector 152 in response to the interaction with the conductive identifier 52 the respective detection signal can be validated by the processor 72 in step 106. Here, the detection signal can be compared with predefined detection signals, e.g. stored in the local storage 75 of the electronic unit 70 so as to identify the electric identifier 50 and thus the type of the medicament container 30 or distal housing component 12.

Typically, the processor 72 performs a data check and evaluates or processes the data or signals obtained from the electric sensor 150. In case that the medicament container 30 is intended for use with this particular injection device 10 the respective data is approved and in step 108 the interlock 90 is controlled or operated by the processor 72 to unlock the drive mechanism 8 of the injection device 10. A user may then commence to use the injection device 10 for setting of a dose and/or for injecting of a dose of the medicament 4. With other situations, and when the medicament container 30 is not intended for use with the injection device 10, the interlock 90 is triggered to block or to lock the drive mechanism 8 of the injection device 10.

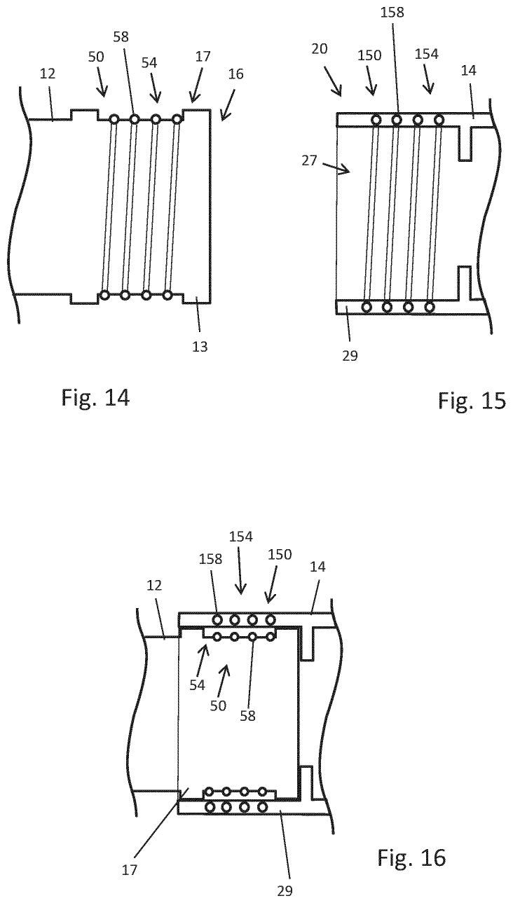

In the example as illustrated in FIGS. 14-16 the electric identifier 50 comprising a helical coil 54 is assembled or arranged on an outside surface of the sidewall 13 of the connecting end 16 of the distal housing component 12. In the illustrated example the coil 54 is provided on an insert portion 17 of the distal housing component 12. With other examples it may be embedded in the sidewall 13. It may be insert molded in the sidewall 13. The helical coil 54 comprises numerous windings 58. The number of windings 58 and/or an inductive response provided by the coil 54 is indicative of a particular distal housing component 12 equipped or provided with a particular medicament.

Here, the number of windings 58 and/or the inductive response of the coil 54 serves to distinguish between numerous available distal housing components 12 and/or numerous medicaments or medicament containers associated therewith. By at least one of the number of windings 58 and the inductive response of the coil 54, as measurable and/or detectable by the electric detector 150, presence of one of a number of different distal housing components 12 as well as presence of one of a dedicated medicament of a number of medicaments located inside the respective distal housing component 12 can be determined.

Furthermore, the electric detector 150 is provided on an inside surface of the sidewall 29 of the proximal housing component 14. The electric detector 150 is provided on or in the sidewall of the connecting end 20 of the proximal housing component 14. The electric detector 150 comprises a helically wound coil 154 with numerous windings 158. The coil 154 may be embedded inside the sidewall 29 of the proximal housing component 14. It may be insert molded in the proximal housing component 14.

The electric detector 150 is provided on or inside the receptacle 27 configured and shaped to receive the insert portion 17 of the distal housing component 12. In the final assembly configuration as illustrated in FIG. 16 there may be provided an annular gap between the coil 54 and the coil 154. In this way, a direct electrical contact between the coils 54 and 154 can be effectively avoided. With some examples, at least one of the coils 54, 154 is embedded inside the sidewall 13, at least one of the distal housing component 12 and the proximal housing component 14. This way, a direct electrical contact between the coils 54, 154 could be even avoided when the outside surface of the insert portion 17 would be in direct mechanical contact with an inside surface of the receptacle 27. With the coils 54, 154 being electrically insulated from each other there can be provided an exclusive inductive coupling between the coils 54, 154 as described above.

In the final assembly configuration as illustrated in FIG. 16, the coil 54 of the electric identifier 50 is arranged inside the coil 154 of the electric detector 150. Such an interleaved or coaxial arrangement is beneficial to obtain and to provide an inductive coupling between the coils 54, 154 and hence between the electric identifier 50 and the electric detector 150.

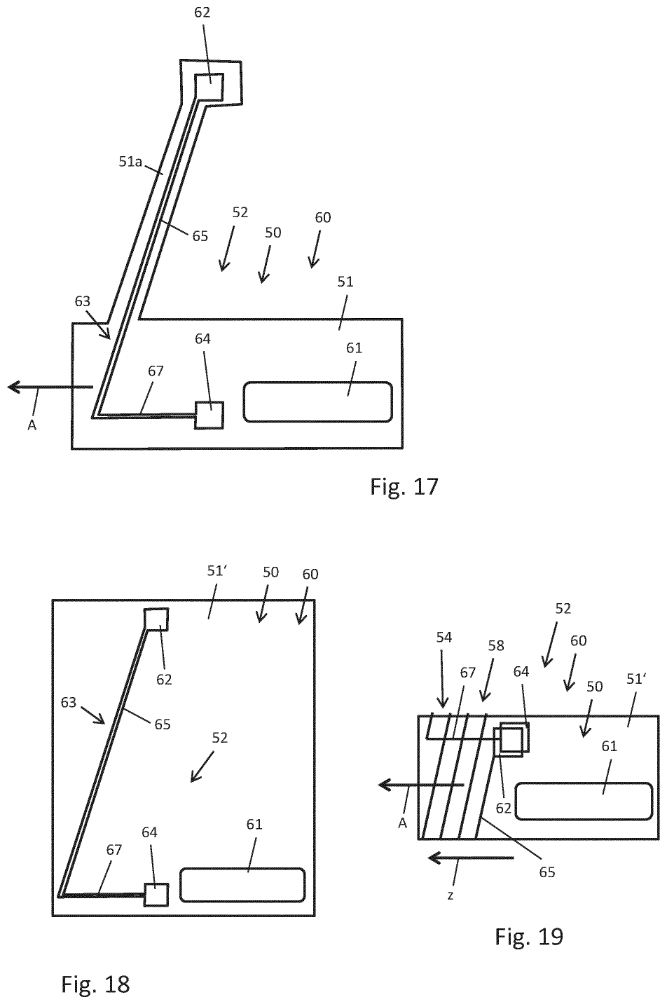

In FIGS. 17-19 further examples of an electronic identifier 50 comprising an inductive coil 54 are illustrated. Here, the coil 54 is formed by a conductor strip 63 provided with a first contact zone 62 at one longitudinal end and a second contact zone 64 at another, e.g. oppositely located longitudinal end of the conductor strip 63. The conductor strip 63 and/or the contact zones 62, 64 may be located, e.g. coated or printed on a planar substrate 51. The planar substrate 51 may comprise a planar sheet. It may form of comprise a label 60 configured for attachment to one of the distal housing component 12, the proximal housing component 14 and/or the planar substrate 51 may be configured for attachment to an outside surface of the medicament container 30.

The label 60 may be provided with a printed label portion 61 on which readable and/or visible information may be provided being indicative about at least one of the distal housing component 12, the proximal housing component 14 or being indicative about information with regard to the container 30 and/or a content thereof. In this way, an electronically readable electronic identifier 50 can be integrated into a label 60 configured for attachment, e.g. adhesive attachment to a housing component 12, 14 of an injection device 10 or to a medicament container thereof 30.

The planar substrate 51 is pliable and/or flexible. It may comprise a flexible foil that can be coiled in a wrapped configuration as indicated in FIG. 19. The substrate 51 and hence the flexible foil may be made of an electrically insulating material. The planar substrate 51 may comprise numerous wraps or windings. In effect, the flexible foil or planar substrate 51 is configured for wrapping around a wrap or coil axis A as indicated in FIG. 17. The wrap or coil axis A may coincide with the longitudinal direction z of one of the distal housing component 12, the proximal housing component 14 and of the medicament container 30, respectively.

As indicated in FIG. 17-19 the conductor strip 63 comprises a first elongated conductor strip portion 65 and a second elongated conductor strip portion 67. The first conductor strip portion 65 extends at an angle with respect to the second conductor strip portion 67. As illustrated in FIGS. 17-19 the second conductor strip portion 67 extends substantially parallel to the wrap axis A. The first conductor strip portion 65 extends at a predefined wrap angle relative to the elongation of the second conductor strip portion 67 and hence relative to the wrap axis A.

When the substrate 51 and hence the label 60 is in the wrapped or coiled configuration with the wrap axis A as an axis of symmetry the first conductor strip portion 65 forms a helical structure as illustrated in FIG. 19. Here, the wrap angle of the first conductor strip portion 65 defines the lead of the helical structure.

Typically, the conductor strip 63 is provided on one of an outside facing surface and an inside facing surface of the wrapped substrate 51 or label 60. Accordingly, the contact zones 62, 64 connected to the free ends of the first and second conductor strip portions 65, 67 are provided on the same side of the substrate 56 or label 60. Typically, the first contact zone 62 is connected with that end of the first conductor strip portion 65 that faces away from the second conductor strip portion 67. The second contact zone 64 is connected with that free end of the second conductor strip portion 67 that faces away from the first conductor strip portion 65. Accordingly, that end of the first conductor strip portion 65 facing away from the first contact zone 62 is directly connected to a respective end of the second conductor strip portion 67 facing away from the second contact zone 64.

The first and second conductor strip portions 65, 67 may be integrally formed and may represent respective sections or portions of a unitary conductor strip 63. Likewise, also the contact zones 62, 64 may be integrally formed or connected with the respective first and second conductor strip portion 65, 67.

With typical examples, the conductor strip 63 as well as the contact zones 62, 64 are provided on the same side of the substrate 51. In this way and when in a coiled or wrapped configuration as illustrated in FIG. 19 the first and second contact zones 62, 64 may at least partially or even completely overlap as seen in a radial direction with regard to the wrap axis A. Here, the first and second contact zones 62, 64 will be electrically insulated by the electrically insulating substrate 51 located between the overlapping contact zones 62, 64.

In this way, the overlapping contact zones 62, 64 may form a capacitor 55 as schematically illustrated in the sketch of FIG. 8. The size and geometry of the contact zones 62, 64 may be individually adapted and designed to provide a capacitor 55 with a capacitance of a predefined size. The contact zones 62, 64 are not intended to provide electrical contact to any other component of the electronic unit 70. Compared to the geometry of the elongated conductor strip 63, the contact zones 62, 64 may comprise a comparatively large surface section of e.g. quadratic, rectangular, round or elliptic shape. A diameter or cross-section through the contact zones 62, 64 is substantially larger than a width of the elongated conductor strip.

When a frequency of an electrical driving signal is in a suitable range for driving the electric detector 150 being inductively coupled with the coil 54, the coil 54 as formed by the conductor strip portions 65, 67 and the mutually overlapping contact zones 62, 64 forms a LC-circuit that provides a distinguishable resonance frequency and hence inductively measurable response.

In effect, the electric identifier 50 is suitable to provide an inductively readable encoding. Different electric identifiers may distinguish by the number of windings of the coil 54 and/or by different capacities of a capacitor 55. Here, and with the example of FIGS. 17-19, the size of the contact zones 62, 64 as well as the material the contact zones 62, 64 are made of and/or the thickness of the substrate 51 as well as the material of the substrate 51 may have a well-defined influence on the size of the capacitance of the capacitor 55 when the contact zones 62, 64 are in an at least partially overlapping configuration.

With some examples, a side of the substrate 51 or label 60 located opposite to that side of the label 60 provided with the conductor strip 63 and/or provided with the contact zones 62, 64 is provided with an adhesive, e.g. with an adhesive layer, configured for attaching the substrate 51 to one of a sidewall of the distal housing component 12, proximal housing component 14 or medicament container 30. With other examples, the adhesive is provided on that side of the substrate 51, which is provided with the conductor strip 63 and/or which is provided with the contact zones 62, 64.

When comparing the example of FIG. 17 with the example of FIG. 18 it is immediately apparent, that the substrate 51′ of the label 60 of FIG. 18 is somewhat larger compared to the substrate 51 of the label 60 as illustrated in FIG. 17. Here, and with the example of FIG. 17 the label 60 or its label portion 61 may not be configured to form multiple windings or wraps. Rather, the substrate 51 as shown in FIG. 17 is provided with a substrate extension 51a protruding from a side edge of the rather rectangularly shaped substrate 51. The shape and geometry of the substrate extension 51a closely corresponds to the geometry and shape of the first conductor strip portion 65 and the associated contact zone 62. Here, it is the substrate extension 51a, which is configured to become wrapped around one of the distal housing component 12, the proximal housing component 14 or the medicament container 30.

1-20. (canceled)

21. An injection device for injecting of a dose of a medicament, the injection device comprising:
a drive mechanism configured to operably engage with a medicament container;
a housing of elongated shape and extending along a longitudinal direction, the housing comprising a distal housing component configured to accommodate the medicament container and a proximal housing component configured to accommodate or to support the drive mechanism to operably engage with the medicament container for expelling or withdrawing of the dose of the medicament from the medicament container;
an electronic unit coupled to the drive mechanism; and
an electric sensor provided at or near a distal end of the proximal housing component, the electric sensor electrically connected to the electronic unit and configured to contactlessly detect an electric identifier provided on or in one of the medicament container and the distal housing component.

22. The injection device of claim 21, wherein the drive mechanism comprises at least one interlock configured to disable at least one of setting of the dose of the medicament and dispensing of the dose of the medicament.

23. The injection device of claim 22, wherein the electronic unit is operable to control the at least one interlock in response to signals received from the electric sensor.

24. The injection device of claim 21, wherein the distal housing component comprises a connecting end for detachably connecting to a connecting end of the proximal housing component.

25. The injection device of claim 24, wherein the electric identifier is arranged on or in one of the connecting end of the distal housing component and a proximal end of the medicament container.

26. The injection device of claim 24, wherein the electric sensor is arranged on or in the connecting end of the proximal housing component.

27. The injection device of claim 21, wherein the electric sensor is configured to contactlessly detect a presence and/or a type of the electric identifier.

28. The injection device of claim 21, wherein the electric identifier comprises an inductive coil or a solenoid coil with a coil axis extending substantially parallel to the longitudinal direction of the housing.

29. The injection device of claim 28, wherein the electric identifier comprises a floating coil.

30. The injection device of claim 28, wherein the inductive coil is provided on a flexible substrate configured for attachment or arrangement to a tubular-shaped sidewall of one of the distal housing component and the medicament container.

31. The injection device of claim 28, wherein the electric identifier is encoded by at least one of a number of windings of the inductive coil, a capacitor electrically connected to the inductive coil, and a microchip electrically connected to the inductive coil.

32. The injection device of claim 21, wherein the electric sensor comprises an inductive coil or a solenoid coil with a coil axis extending substantially parallel to the longitudinal direction of the housing.

33. The injection device of claim 21, wherein the electric identifier comprises a passive electronic identifier.

34. The injection device of claim 33, wherein the passive electronic identifier comprises a near field communication (NFC) tag configured to communicate with the electric sensor implemented as a corresponding near field communication interface of the electronic unit.

35. The injection device of claim 33, wherein the passive electronic identifier comprises a non-volatile memory to store at least one of a medicament name, a medicament manufacturing date, a medicament expiry date, a lot number of the medicament, and a unique medicament container ID.

36. The injection device according to claim 35, wherein the non-volatile memory is at least one of erasable or writable by the near field communication interface.

37. The injection device of claim 22, comprising a first component and a second component, the first component is movable relative to the second component for at least one of setting of the dose and injecting of the dose;
wherein the at least one interlock comprises an electromechanical interlock configured to mechanically engage with the first component and with the second component; and
wherein the at least one interlock is operable to switch between an interlocked state, in which the first component is movably locked to the second component, and a release state, in which the first component is movable relative to the second component.

38. The injection device of claim 22, wherein the drive mechanism comprises at least one clutch operable to switch the drive mechanism between a dose setting mode and a dose injecting mode; and
wherein the at least one interlock is operably engaged with the at least one clutch to selectively disable switching of the drive mechanism from one of the dose setting mode and the dose injecting mode into the other one of the dose setting mode and the dose injecting mode.

39. The injection device of claim 21, wherein the medicament container filled with the medicament is arranged inside the housing.

40. A method of unlocking operation of an injection device for at least one of setting and injecting of a dose of a medicament, wherein the injection device comprises a drive mechanism configured to operably engage with a medicament container, a housing comprising a distal housing component configured to accommodate the medicament container and a proximal housing component configured to accommodate or to support the drive mechanism, an electronic unit coupled to the drive mechanism, an electric sensor electrically connected to the electronic unit and configured to detect an electric identifier provided on or in one of the medicament container and the distal housing component, and at least one interlock configured to disable at least one of setting of the dose and dispensing of the dose, wherein the method comprises:
detecting at least one of a presence and a type of an electric identifier by the electric sensor;
generating at least one detection signal indicative of the electric identifier and transmitting the at least one detection signal to the electronic unit;
validating the at least one detection signal; and
at least one of temporarily deactivating or re-activating the at least one interlock based on a validation of the at least one detection signal.
